(19) United States
(12) Patent Application Publication
(10) Pub. No.: US 2007/0185337 A1
(43) Pub. Date: Aug. 9, 2007

(54) Process for the preparation of optically pure 4-hydroxy-2-oxo-1-pyrrolidine acetamide

(75) Inventors: Seong-Jin Kim, Deajeon (KR); Chung-woo Lim, Seoul (KR); Chang Jin Boo, Jeju-do (KR); Jung-hoon Oh, Chungcheongnam-do (KR); Ki Hyun Kim, Incheon (KR); Jae Kwan Lee, Incheon (KR); Duk Kwon Won, Gyeonggi-do (KR)

Correspondence Address: MORGAN LEWIS & BOCKIUS LLP, 1111 PENNSYLVANIA AVENUE NW, WASHINGTON, DC, 20004, US

(73) Assignees: AHN-GOOK PHARMACEUTICAL CO., LTD., Seoul (KR); RSTECH CORPORATION, Daejeon (KR)

(21) Appl. No.: 11/596,580

(22) PCT Filed: May 25, 2005
(86) PCT No.: PCT/KR05/01535

§ 371 Date: Nov. 15, 2006

(30) Foreign Application Priority Data
May 25, 2004 (KR) 10-2004-0037320

(51) Int. Cl. C07D 207/04

(52) U.S. Cl. 548/546

(57) ABSTRACT

A process for the preparation of chiral 4-hydroxy-2-oxo-1-pyrrolidine acetamide includes adding sodium cyanide together with citric acid to a solution of chiral epichlorohydrin to obtain chiral 3-chloro-2-hydroxypropionitrile by ring opening reaction of the chiral epichlorohydrin, reacting the obtained product with an alcohol containing hydrochloride gas to obtain chiral 4-chloro-3-hydroxybutyric acid ester, and reacting the obtained product in a presence of a base with glycinamide or with glycine ester accompanied by ammonolysis with ammonia to produce the targeted chiral 4-hydroxy-2-oxo-1-pyrrolidine acetamide.

This application is a U.S. National Phase Application of International Application PCT/KR2005/001535, filed May 25, 2005, which claims the benefit of Korean Patent Application No. 10-2004-0037320, filed May 25, 2004, which are hereby incorporated by reference in their entirety.

### TECHNICAL FIELD

The present invention relates to a process for the preparation of optically pure 4-hydroxy-2-oxo-1-pyrrolidine acetamide. More specifically, the present invention relates to a process for the preparation of 4-hydroxy-2-oxo-1-pyrrolidine acetamide with high purity and in high yield, comprising obtaining 3-chloro-2-hydroxypropionitrile by epoxy ring opening of chiral epichlorohydrin and reacting the obtained product with an alcohol containing hydrochloride gas to give 4-chloro-3-hydroxybutyric acid ester, followed by reaction with glycinamide, or subsequently with glycine ester and ammonia.

### BACKGROUND ART

4-Hydroxy-2-oxo-1-pyrrolidine acetamide (commercially called as “oxyracetam”), represented by formula 1, is a cardiovascular drug for use as a brain function enhancer or in deliberating dementia such as Alzheimer or multi-infarctual dementia: 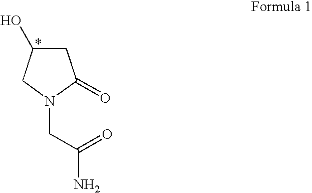
wherein the asterisk represents a chiral center.

In the syndromes to which the oxyracetam can be applicable, a compound having superior efficacy to the oxyracetam was not found. For this reason, the oxyracetam has been widely used in a market. Even though (R)-enantiomer and (S)-enantiomer do not exhibit equivalent efficacy, the compound has been used in a racemate form. The reason is believed to be that the process which provides optically pure chiral compound with high purity was not commercially available.

Conventional methods for the preparation of 4-hydroxy-2-oxo-1-pyrrolidine acetamide having formula 1 are as follows:

U.S. Pat. Nos. 4,824,966, 4,843,166 and 5,276,164 issued to Lonza Ltd. discloses a process for the preparation of the oxyracetam and intermediates thereof. The process disclosed in the patents comprises reacting 4-(C₁-C₂)-alkoxy-3-pyrrolin-2-on-1-yl-acetic acid (C₁-C₄)-alkyl ester with trichloromethylsilane to protect hydroxyl group, followed by hydrogenation and amidation of the obtained product. According to the process, a racemic oxyracetam is obtained from reduction of double bond by hydrogenation. Therefore, the process suffers from the disadvantage that it is not applicable to the preparation of optically pure oxyracetam. Further, preparation of 4-(C₁-C₂)-alkoxy-3-pyrrolin-2-on-1-yl-acetic acid (C₁-C₄)-alkyl ester has a low yield.

U.S. Pat. Nos. 4,124,594, 4,173,569 and 4,629,797 issued to I.S.F. Spa discloses a process for the preparation of optically pure oxyracetam. The process disclosed in the patents comprises reacting optically pure (S)-γamino-β-hydroxybutyric acid with a silylating agent to protect a hydroxyl group, reacting the obtained product with a halogen compound of an aliphatic acid of the formula Hal (CH₂COOR), in which Hal represents a halogen atom, in the presence of a acid receptor, followed by cyclization and hydrolysis to provide the optically pure oxyracetam. While the method provides the optically pure oxyracetam, it suffers from disadvantages: expensive starting material, low yield due to multi-steps, and high cost.

Another alternative process for the preparation of the oxyracetam is disclosed in U.S. Pat. No. 4,797,496 and WO 93/06826. The process disclosed in the documents comprises obtaining chiral alkyl 3,4-epoxybutanoate from a chiral β-hydroxybutyrolactone, reacting the obtained product with N-protected glycinamide, and N-deprotecting the obtained product followed by cyclization to give the optically pure oxyracetam. This method has shorter steps than those of U.S. Pat. No. 3,124,594 and its related patents. However, this method suffers from high cost, due to very poor yield in the synthesis of chiral alkyl 3,4-epoxybutanoate.

U.S. Pat. No. 4,686,296 owned by Denki Kagaku Kogyo Kabushiki Kaisha discloses a process for the preparation of optically pure (S)-oxyracetam, comprising one step of reacting halohydroxy butyrate or epoxy butyrate with glycinamide to produce said oxyracetam. In the process, the most important key point is how chiral 4-halo-3-hydroxybutyrate, which is a starting material of the process, can be secured.

Korean Patent Publication No. 2000-9465 filed by Samsung Chemical discloses a process for the preparation of the optically pure (S)-oxyracetam. In the process, (S)-3,4-epoxybutyric acid salt is firstly synthesized as an intermediate in an aqueous condition from optically pure (S)-3-hydroxybutyrolactone. And then, the intermediate compound is subjected to amidation with glycinamide in an aqueous condition, accompanied by cyclization. Although this technology seems to be an industrially advantageous one over the processes mentioned in the above in terms of the yield and the purity, it also suffers from disadvantages that many impurities are produced due to low purity of (S)-3-hydroxybutyrolactone, and that preparation of highly pure (S)-3-hydroxybutyrolactone is not yet accomplishable. As a result, the process does not give an oxyracetam having the purity suitable for drug application.

Besides, the processes filed by Binex, Hwail Pharmaceuticals and Korea Research Institute of Chemical Technology are also known as Korean Patent Publication Nos. 2003-83466, 2003-48746 and 2003-42883. The processes of the documents aim at producing a racemic oxyracetam. Even though chiral compound might be prepared based on the processes, raw materials would be expensive and not commercially available, and the processes would be not applicable due to a low competitive price.

### DISCLOSURE OF INVENTION

### Technical Problem

Extensive Researches by the present inventors to avoid the problems mentioned above reveal that techniques for preparing chiral key intermediates are essential for the effective preparation of the chiral 4-hydroxy-2-oxo-1-pyrrolidine acetamide of formula 1. Therefore, in the present invention, the chiral 3′-hydroxypropionitrile which is an important key intermediate is produced in a safe and economic manner and in an industrial scale. More specifically, the chiral 3′-hydroxypropionitrile is produced with high purity and in high yield by reaction of chiral epichlorohydrin with citric acid and sodium cyanide, which is one of improvements to the conventional techniques. Thereafter, the chiral 3′-hydroxypropionitrile is converted to chiral 4-chloro-3-hydroxybutyric acid ester, followed by reaction with glycinamide or subsequently with glycine ester and ammonia. Throughout the successive reactions, the chiral oxyracetam with high optical purity and high chemical purity is obtained.

Therefore, an object of the present invention is to provide a process for the preparation of the chiral oxyracetam with high purity, in a safe and economic manner and in an industrial scale.

### TECHNICAL SOLUTION

The above object and other objects which will be described in the detailed description of the specification can be accomplished by provision of a process for the preparation of chiral 4-hydroxy-2-oxo-1-pyrrolidine acetamide, comprising:

(a) adding sodium cyanide together with citric acid to a solution of chiral epichlorohydrin to obtain chiral 3-chloro-2-hydroxypropionitrile by ring opening reaction of the chiral epichlorohydrin;

(b) reacting the obtained product with an alcohol containing hydrochloride gas to obtain chiral 4-chloro-3-hydroxybutyric acid ester; and

(c) reacting the obtained product in a presence of a base with glycinamide or with glycine ester accompanied by ammonolysis with ammonia to produce the targeted chiral 4-hydroxy-2-oxo-1-pyrrolidine acetamide.

### ADVANTAGEOUS EFFECTS

The process according to the present invention produces chiral 3-chloro-2-hydroxypropionitrile by the treatment of citric acid and sodium cyanide that are no harmful and easily treatable, and then the targeted 4-hydroxy-2-oxo-1-pyrrolidine acetamide with high purity and in high yield. The process is suitable for industrial application. Therefore, the process according to the present invention is useful for the preparation of chiral 4-hydroxy-2-oxo-1-pyrrolidine acetamide that is used as a cerebrovascular drug.

### MODE FOR THE INVENTION

According to the present invention, there is provided a process for the preparation of optically pure 4-hydroxy-2-oxo-1-pyrrolidine acetamide, comprising:

(a) adding sodium cyanide together with citric acid to a solution of chiral epichlorohydrin of formula 2 to obtain chiral 3-chloro-2-hydroxypropionitrile of formula 3 by ring opening reaction of the chiral epichlorohydrin;

(b) reacting the obtained product with an alcohol containing hydrochloride gas to obtain chiral 4-chloro-3-hydroxybutyric acid ester of formula 4; and

(c) reacting the obtained product in a presence of a base with glycinamide or with glycine ester accompanied by ammonolysis with ammonia to produce the targeted chiral 4-hydroxy-2-oxo-1-pyrrolidine acetamide. 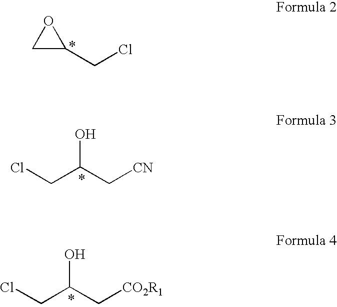
In the Formula 2 to 4, the asterisk represents a chiral center and R₁ represents an alkyl group.

The process of the present invention can be summarized in a reaction scheme 1: 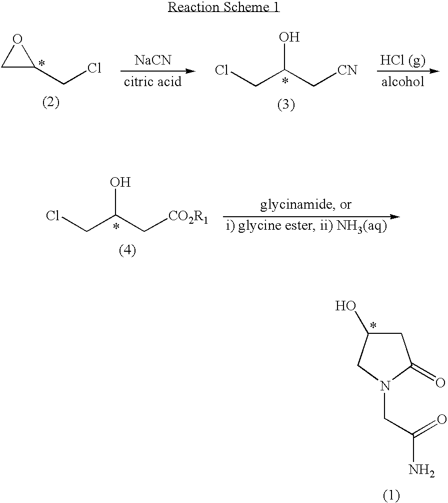
wherein the asterisk represents a chiral center and R₁ represent an alkyl group.

As shown in the reaction scheme 1, the present invention uses as a starting material a chiral epoxy compound, specifically a chiral epichlorohydrin of formula 2. The chiral epichlorohydrin is obtained from chiral resolution of racemic epichlorohydrin. Particularly, the compound is obtained by reacting the racemic epichlorohydrin in a presence of a chiral catalyst with a nucleophile and isolating un-reacted isomer from a reaction mixture. Preferably, the compound is obtained by subjecting the racemic epichlorohydrin in the presence of a chiral catalyst to hydrolysis resolution and isolating un-reacted isomer from a reaction mixture. With regard to more detailed explanation, Please refer to Korean Patent Nos. 319045, 342659 and 368002, U.S. Pat. Nos. 5,665,890, 5,929,232 6,262,278 and 6,720,434, and European Patent No. 1,292,602.

The chiral epichlorohydrin of formula 2 undergoes ring opening reaction by a cyanide group. Various methods are known to accomplish ring opening of the chiral epoxy compound with the cyanide group: HCN as the cyanide group [Bull. Soc. Chim. Fr. 3, 138(1936); Bull. Acad. R. Belg. 29, 256(1943); Ber., 12, 23(1879); and Japanese Patent Publication H1 1-39559]; a combination of a cyanide salt and acetic acid in a water solvent to maintain pH at a range of 8.0-10.0 [Japanese Patent Publication S63-316758]; and a combination of a cyanide salt and an inorganic acid to maintain pH at a range of 8.0-10.0 [Japanese Patent Publication H5-310671]. However, those methods suffer from one or more disadvantages: a poor working environment; a low yield; and a low optical purity. As a result, those are not applicable to an industrial mass-production. As shown in the reaction scheme 1, the present invention avoids the problems by adopting sodium cyanide in combination with citric acid. The citric acid is a tri-acid having three carboxyl groups and easily dissolves into a water solvent so that it can be used as a concentrated solution, which gives another industrial advantage. Further, the citric acid has no reactivity with the targeted product obtained from the ring opening reaction, thereby producing no byproduct which might be produced from the reaction of the citric acid with the targeted product. According to the preferred specific embodiment of the present invention, ring opening was performed by adding the sodium cyanide together with the citric acid to the chiral epichlorohydrin dissolved into a water solvent at a range of pH 7.8-8.3.

As a result of the ring opening reaction of the chiral epichlorohydrin having formula 2, chiral 3-chloro-2-hydroxypropionitrile is produced in a mild condition, in high yield and with high optical purity. Thereafter, the obtained product reacts with an alcohol containing hydrochloride gas to give chiral 4-chloro-3-hydroxybutyric acid ester of formula 4. The compounds having formula 3 and 4, produced from the epoxy compound, are valuable raw material as intermediates for the preparation of a drug. Further, syntheses of the compounds are performed in a mild condition, in high yield and with high optical purity, which is suitable for industrial application. Therefore, the compounds 3 and 4, and their syntheses are basis for the preparation of the targeted compound in an easy, commercially available route and in a cost-effective manner. Preferred Examples of the alcohol into which hydrochloride gas was dissolved are alcohols having 1 to 4 carbon atoms. Specifically, methanol, ethanol, propanol, isopropanol, butanol, isobutanol and t-butanol may be used. Regarding toxicity, handling and yield, ethanol is most preferable.

The chiral 4-chloro-3-hydroxybutyric acid ester of formula 4 give the targeted compound of formula 1, chiral 4-hydroxy-2-oxo-1-pyrrolidine acetamide, in a presence of a base, by reaction with glycinamide or by reaction with glycine ester and subsequent ammonolysis with ammonia, which is summarized in reaction scheme 2: 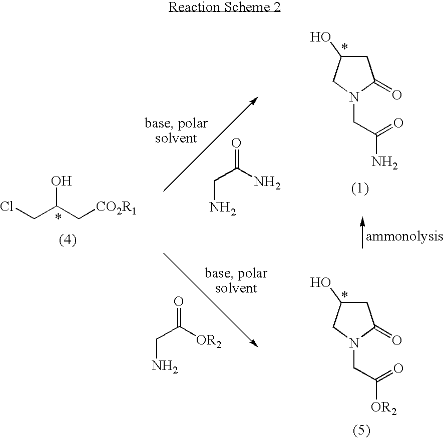
wherein R₁ and R₂ each independently represent alkyl groups and the asterisk represents a chiral center.

The reaction of the chiral 4-chloro-3-hydroxybutyric acid ester having formula 4 with glycinamide comprises substitution of a chloride atom of the chiral 4-chloro-3-hydroxybutyric acid ester by an amino group of the glycinamide, and subsequent cyclization by intramolecular condensation with the amino group to a carbonyl group of the chiral 4-chloro-3-hydroxybutyric acid ester. Herein, the reaction is performed in a presence of a base and a polar solvent. As a base, sodium carbonate, sodium bicarbonate, potassium carbonate, sodium hydroxide and potassium hydroxide may be mentioned. As a polar solvent, methanol, ethanol, acetonitrile and tetrahydrofuran may be mentioned. The glycinamide is added typically in a form of salt, preferably in a form of HCl salt. A reaction temperature can be suitably chosen in a range of 0° C.-100° C., and a stirring time in a range of 1 to 20 hours.

When glycine ester is used instead of glycinamide, reaction proceeds in the same pathway. Specifically, the chloride group of the chiral 4-chloro-3-hydroxybutyric acid ester is substituted with the amino group of the glycinamide, and subsequent intramolecular condensation with the amino group to the carbonyl group yields cyclization to give chiral 4-hydroxy-2-oxo-1-pyrrolidine ester of formula 5. The reaction is performed in a presence of a base and a polar solvent. The base and the solvent mentioned above can be used. Preferred examples of the glycine ester are glycine (C₁-C₄)-alkyl esters. Glycine ethyl ester or glycine methyl ester is particularly preferable. The obtained product provides the targeted compound of formula 1, chiral 4-hydroxy-2-oxo-1-pyrrolidine acetamide, by ammonolysis with an aqueous ammonia.

The process according to the present invention provides optically pure (R)-oxyracetam or (S)-oxyracetam. Preferable is (S)-oxyracetam. Conventional processes use expensive or industrially inapplicable chiral raw materials. To the contrary, the present invention adopts, as a starting material, chiral epichlorohydrin that is inexpensive and commercially producible in high optical purity, and uses a combination of sodium cyanide and citric acid to result in the ring opening which produces 3-chloro-2-hydroxypropionitrile of formula 3 in an economic and industrially applicable manner. Thereafter, the obtained product undergoes a reaction with an alcohol containing HCl (g) to produce 4-chloro-3-hydrobutyric acid ester of formula 4, and then a reaction with glycinamide or a subsequently with glycine ester and ammonia to produce the targeted compound of formula 1, as shown in the reaction scheme 2. The process according to the present invention is a simple and enhanced one in terms of procedures and purity, compared to the conventional processes.

In the following, the present invention will be more fully illustrated referring to Examples, but it should be understood that these Examples are suggested only for illustration and should not be construed to limit the scope of the present invention.

### EXAMPLE 1

### Preparation of (R)-3-chloro-2-hydroxypropionitrile

To 5 L of a 3-necked round bottom flask equipped with a thermometer, a pH meter and a stirrer, 400 g of water and 400 g of (R)-epichlorohydrin were successively added. To the stirred solution, 275 g of sodium cyanide dissolved into 347 g of water and 427 g of citric acid dissolved into 347 g of water were simultaneously and dropwisely added. The pH and the temperature of the reaction solution were maintained at a range of 7.8-8.3 and 25° C.-8.3° C., respectively. After dropwise addition, the temperature was raised to a room temperature and further stirred for 10 hours. The reaction mixture was extracted with 2 L (×2) of ethyl acetate, and the organic layers were collected and dried with anhydrous magnesium sulfate. After filtration, the filtrate was evaporated under reduced pressure to give the targeted chiral 3-chloro-2-hydroxypropionitrile.

### EXAMPLE 2

### Preparation of (S)-3-chloro-2-hydroxypropionitrile

To 5 L of a 3-necked round bottom flask equipped with a thermometer, a pH meter and a stirrer, 400 g of water and 400 g of (S)-epichlorohydrin were successively added. To the stirred solution, 275 g of sodium cyanide dissolved into 347 g of water and 427 g of citric acid dissolved into 347 g of water were simultaneously and dropwisely added. The pH and the temperature of the reaction solution were maintained at a range of 7.8-8.3 and 25° C.-8.3° C., respectively. After dropwise addition, the temperature was raised to a room temperature and further stirred for 10 hours. To the reaction mixture, 200 g of brine was added. The reaction mixture was distributed into 5 L of ethyl acetate, and the ethyl acetate layer was separated. To the ethyl acetate solution, 50 g of anhydrous sodium sulfate was added and stirred for 30 minutes. After filtration, the filtrate was evaporated under reduced pressure. The concentrated solution was distilled using an agitated film evaporator (110° C./lmbar) to give 456 g of the targeted (S)-3-chloro-2-hydroxypropionitrile.

¹H NMR (CDCl₃, 300 MHz, TMS as an internal standard) δ 2.80 (d, 2H, J=5 Hz), 3.2-3.68 (m, 1H), 3.66 (d, 2H, J=6 Hz), 4.08-4.22 (m, 1H).

### EXAMPLE 3

### Preparation of methyl-(S)-4-chloro-3-hydroxybutyric acid

To 3 L of a 3-necked round bottom flask equipped with a thermometer, a pH meter and a stirrer, 439 g of methyl alcohol was added and cooled to −20° C. To the solution, 372 g of hydrochloride gas was supplied. Maintaining the temperature to −5° C.-0° C., 458 g of (S)-3-chloro-2-hydroxypropionitrile was dropwisely added. After dropwise addition, the reaction temperature was raised to 20° C.-25° C. and stirred for 12 hours. The reaction mixture was evaporated under reduced pressure to remove the methyl alcohol. To the residue, 664 g of water was added and stirred for 1 hour. And then, the aqueous solution was extracted with 1.5 L (×2) of ethyl acetate, and the organic layers were collected and dried with anhydrous magnesium sulfate. After filtration, the filtrate was evaporated under reduced pressure. Fractional distillation to the residue gave 342 g of the targeted methyl-(S)-4-chloro-3-hydroxybutyric acid.

¹H NMR (CDCl₃, 300 MHz, TMS as an internal standard) δ 2.35-2.42 (m, 2H), 3.17 (d, 1H, J=5 Hz), 3.66 (s, 3H), 3.51-3.57 (m, 2H), 4.20-4.30 (m, 1H).

### EXAMPLE 4

### Preparation of ethyl-(S)-4-chloro-3-hydroxybutyric acid

To 3 L of a 3-necked round bottom flask equipped with a thermometer, a pH meter and a stirrer, 631 g of ethyl alcohol was added and cooled to −20° C. To the solution, 372 g of hydrochloride gas was supplied. Maintaining the temperature to −5° C.-0° C., 458 g of (S)-3-chloro-2-hydroxypropionitrile was dropwisely added. After dropwise addition, the reaction temperature was raised to 20° C.-25° C. and stirred for 12 hours. Work-up procedures were performed in the same manner as mentioned in the Example 3.490 g of the targeted ethyl-(S)-4-chloro-3-hydroxybutyric acid was obtained.

¹H NMR (CDCl₃, 300 MHz, TMS as an internal standard) δ 1.28 (t, 3H, J=5 Hz), 2.55-2.70 (m, 2H), 3.17 (d, 1H, J=5 Hz), 3.55-3.65 (m, 2H), 4.18 (q, 2H, J=7.4 Hz), 4.17-4.20 (m, 1H).

### EXAMPLE 5

### Preparation of propyl-(S)-4-chloro-3-hydroxybutyric acid

To 3 L of a 3-necked round bottom flask equipped with a thermometer, a pH meter and a stirrer, 823 g of propyl alcohol was added and cooled to −20° C. To the solution, 372 g of hydrochloride gas was supplied. Maintaining the temperature to −5° C.-0° C., 458 g of (S)-3-chloro-2-hydroxypropionitrile was dropwisely added. After dropwise addition, the reaction temperature was raised to 20° C.-25° C. and stirred for 12 hours. Work-up procedures were performed in the same manner as mentioned in the Example 3.620 g of the targeted propyl-(S)-4-chloro-3-hydroxybutyric acid was obtained.

### EXAMPLE 6

### Preparation of isopropyl-(S)-4-chloro-3-hydroxybutyric acid

To 3 L of a 3-necked round bottom flask equipped with a thermometer, a pH meter and a stirrer, 823 g of isopropyl alcohol was added and cooled to −20° C. To the solution, 372 g of hydrochloride gas was supplied. Maintaining the temperature to −5° C.-0° C., 458 g of (S)-3-chloro-2-hydroxypropionitrile was dropwisely added. After dropwise addition, the reaction temperature was raised to 20° C.-25° C. and stirred for 12 hours. Work-up procedures were performed in the same manner as mentioned in the Example 3. 611 g of the targeted isopropyl-(S)-4-chloro-3-hydroxybutyric acid was obtained.

### EXAMPLE 7

### Preparation of (S)-4-hydroxy-2-oxo-1-pyrrolidine acetamide

To 1 L of a 3-necked round bottom flask equipped with a thermometer, a pH meter and a stirrer, 64.8 g of glycinamide hydrochloride, 124.3 g of sodium carbonate and 500 mL of ethyl alcohol were successively added and stirred at a room temperature for 1 hour. To the solution, 97.7 g of ethyl-(S)-4-chloro-3-hydroxybutyric acid obtained in the above was dropwisely added. The reaction solution was further stirred at 80° C. for 20 hours. The hot reaction mixture was filtered to remove precipitate and washed with 50 mL of ethyl alcohol. The filtrate was evaporated under reduced pressure. The residue was dissolved into 120 g of water and the aqueous solution was washed with 120 g of dichloromethane. The aqueous layer was concentrated under reduced pressure. The residue was dissolved into 30 mL of methyl alcohol. Column chromatography of 20 g of silica gel (eluent: dichloromethane containing 20% methyl alcohol) was performed. The collected solution was evaporated under reduced pressure, and recrystallization with methyl alcohol and acetone gave 60.3 g of the targeted (S)-4-hydroxy-2-oxo-1-pyrrolidine acetamide in high purity.

¹H NMR (DMSO-d₆, 300 MHz) δ 2.10 (d, 1H, J=16.9 Hz), 2.57 (dd, 1H, J=9.6, J=5.5 Hz), 3.69 (d, 1H, J=16.6 Hz), 3.88 (d, 1H, J=16.6 Hz), 2.10 (d, 1H, J=16.9 Hz), 4.31 (m, 1H), 5.25 (s, H), 7.13 (s, 1H), 7.33 (s, 1H).

### EXAMPLE 8

### Preparation of (S)-4-hydroxy-2-oxo-1-pyrrolidine acetamide

(S)-4-hydroxy-2-oxo-1-pyrrolidine acetamide was prepared in the same manner as described in the Example 7 except that 89.5 g of methyl-(S)-4-chloro-3-hydroxybutyric acid was used instead of 97.7 g of ethyl-(S)-4-chloro-3-hydroxybutyric acid. 57.8 g of the targeted compound was obtained.

### EXAMPLE 9

### Preparation (S)-4-hydroxy-2-oxo-1-pyrrolidine acetamide

(S)-4-hydroxy-2-oxo-1-pyrrolidine acetamide was prepared in the same manner as described in the Example 7 except that 105.9 g of propyl-(S)-4-chloro-3-hydroxybutyric acid was used instead of 97.7 g of ethyl-(S)-4-chloro-3-hydroxybutyric acid. 50.3 g of the targeted compound was obtained.

### EXAMPLE 10

### Preparation of (S)-4-hydroxy-2-oxo-1-pyrrolidine acetamide

(S)-4-hydroxy-2-oxo-1-pyrrolidine acetamide was prepared in the same manner as described in the Example 7 except that 105.9 g of isopropyl-(S)-4-chloro-3-hydroxybutyric acid was used instead of 97.7 g of ethyl-(S)-4-chloro-3-hydroxybutyric acid. 47.9 g of the targeted compound was obtained.

### EXAMPLE 11

### Preparation of (S)-4-hydroxy-2-oxo-1-pyrrolidine acetamide

To 1 L of a 3-necked round bottom flask equipped with a thermometer, a pH meter and a stirrer, 64.8 g of glycinamide hydrochloride, 98.5 g of sodium bicarbonate and 500 mL of ethyl alcohol were successively added and stirred at a room temperature for 1 hour. To the solution, 97.7 g of ethyl-(S)-4-chloro-3-hydroxybutyric acid was dropwisely added. The reaction solution was further stirred at 80° C. for 20 hours. Work-up procedures were performed in the same manner as mentioned in the Example 7. Recrystallization with methyl alcohol and acetone gave 52.5 g of the targeted (S)-4-hydroxy-2-oxo-1-pyrrolidine acetamide.

### EXAMPLE 12

### Preparation of (S)-4-hydroxy-2-oxo-1-pyrrolidine acetamide

To 1 L of a 3-necked round bottom flask equipped with a thermometer, a pH meter and a stirrer, 64.8 g of glycinamide hydrochloride, 162 g of potassium carbonate and 500 mL of ethyl alcohol were added and stirred at a room temperature for 1 hour. To the solution, 97.7 g of ethyl-(S)-4-chloro-3-hydroxybutyric acid was dropwisely added. The reaction solution was further stirred at 80° C. for 20 hours. Work-Lip procedures were performed in the same manner as mentioned in the Example 7. Recrystallization with methyl alcohol and acetone gave 58.3 g of the targeted (S)-4-hydroxy-2-oxo-1-pyrrolidine acetamide.

### EXAMPLE 13

### Preparation of (S)-4-hydroxy-2-oxo-1-pyrrolidine acetethyl ester

To 1 L of a 3-necked round bottom flask equipped with a thermometer, a pH meter and a stirrer, 81.8 g of glycine ethyl ester, 124.3 g of sodium carbonate and 500 mL of ethyl alcohol were successively added and stirred at a room temperature for 1 hour. To the solution, 97.7 g of ethyl-(S)-4-chloro-3-hydroxybutyric acid was dropwisely added. The reaction solution was further stirred at 80° C. for 20 hours. The hot reaction mixture was filtered to remove precipitate and washed with 50 mL of ethyl alcohol. The filtrate was evaporated under reduced pressure. The residue was dissolved into 30 mL of methyl alcohol. Column chromatography of 20 g of silica gel (eluent: dichloromethane containing 20% methyl alcohol) gave 68.4 g of the targeted (S)-4-hydroxy-2-oxo-1-pyrrolidine acetethyl ester.

¹H NMR (CDCl₃, 300 MHz, TMS as an internal standard) δ 1.28(t, 3H, J=7.2 Hz), 2.38 (dd, 1H, J=17.5, J=2.5 Hz), 2.69 (dd, 1H, J=17.4, J=6.5 Hz), 3.34 (dd, 1H, J=10.4, J=1.9 Hz), 3.77 (dd, 1H, J=10.4, J=5.6 Hz), 3.93 (d, 1H, J=17.5 Hz), 4.18 (d, 1H, J=17.5 Hz), 4.19 (q, 2H, J=7.2 Hz), 4.30 (bs, 1H), 4.50 (m, 1H).

### EXAMPLE 14

### Preparation of (S)-4-hydroxy-2-oxo-1-pyrrolidine acetamide

To 1 L of a 3-necked round bottom flask equipped with a thermometer, a pH meter and a stirrer, 73 g of (S)-4-hydroxy-2-oxo-1-pyrrolidine acetethyl ester was added. After addition of 73 mL of 30% aqueous ammonia at 0° C., the temperature of the reaction solution was raised to 20° C. and stirred for 20 hours. The reaction mixture was concentrated under reduced pressure. To the concentrate, 100 mL of ethyl alcohol was added in order to remove the remaining water through azeotropic technique. Recrystallization with methyl alcohol and acetone gave 51.2 g of the targeted (S)-4-hydroxy-2-oxo-1-pyrrolidine acetamide in high purity.

1. A process for the preparation of chiral 4-hydroxy-2-oxo-1-pyrrolidine acetamide, comprising:
adding sodium cyanide together with citric acid to a solution of chiral epichlorohydrin to obtain chiral 3-chloro-2-hydroxypropionitrile by ring opening reaction of the chiral epichlorohydrin;
reacting the obtained product with an alcohol containing hydrochloride gas to obtain chiral 4-chloro-3-hydroxybutyric acid ester; and
reacting the obtained product in a presence of a base with glycinamide or with glycine ester accompanied by ammonolysis with ammonia to produce the targeted chiral 4-hydroxy-2-oxo-1-pyrrolidine acetamide.

2. The process as set forth in claim 1, wherein the step (a) is performed in an aqueous system and pH is maintained at a range of 7.8-8.3.

3. The process as set forth in claim 1, wherein the chiral epichlorohydrin is obtained from chiral resolution.

4. The process as set forth in claim 1, wherein the chiral epichlorohydrin is obtained from hydrolyzing a racemic epichlorohydrin by reaction with water and isolating un-reacted isomer form a reaction mixture.

5. The process as set forth in claim 1, wherein the chiral 4-hydroxy-2-oxo-1-pyrrolidine acetamide is optically active.

6. The process as set forth in claim 1, wherein the chiral 4-hydroxy-2-oxo-1-pyrrolidine acetamide is (S)-isomer.

7. The process as set forth in claim 1, comprising (a) adding sodium cyanide together with citric acid to a solution of chiral epichlorohydrin of formula 2 to obtain chiral 3-chloro-2-hydroxypropionitrile of formula 3 by ring opening reaction of the chiral epichlorohydrin, (b) reacting the obtained product with an alcohol containing hydrochloride gas to obtain chiral 4-chloro-3-hydroxybutyric acid ester of formula 4, and (c) reacting the obtained product in a presence of a base with glycinamide to produce the targeted chiral 4-hydroxy-2-oxo-1-pyrrolidine acetamide of formula 1, which is shown in a reaction scheme 3: 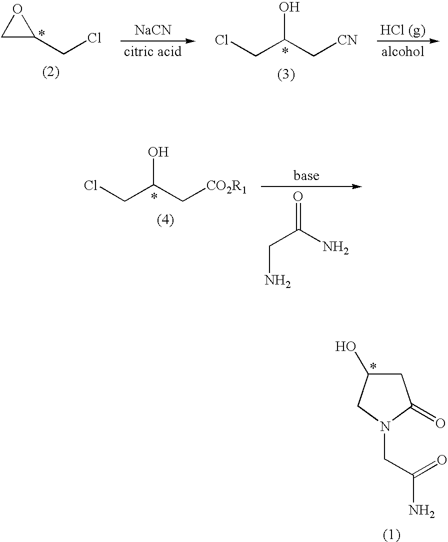
wherein R₁ represents an alkyl group having 1 to 4 carbon atoms and the asterisk represents a chiral center.

8. The process as set forth in claim 7, wherein R₁ is selected from the group consisting of methyl, ethyl, propyl, isopropyl, butyl, isobutyl and t-butyl.

9. The process as set forth in claim 8, wherein R₁ is ethyl.

10. The process as set forth in claim 1, comprising (a) adding sodium cyanide together with citric acid to a solution of chiral epichlorohydrin of formula 2 to obtain chiral 3-chloro-2-hydroxypropionitrile of formula 3 by ring opening reaction of the chiral epichliorohydrin, (b) reacting the obtained product with an alcohol containing hydrochloride gas to obtain chiral 4-chloro-3-hydroxybutyric acid ester of formula 4, and (c) reacting the obtained product in a presence of a base with glycine ester accompanied by ammonolysis with ammonia to produce the targeted chiral 4-hydroxy-2-oxo-1-pyrrolidine acetamide of formula 1, which is shown in reaction scheme 4: 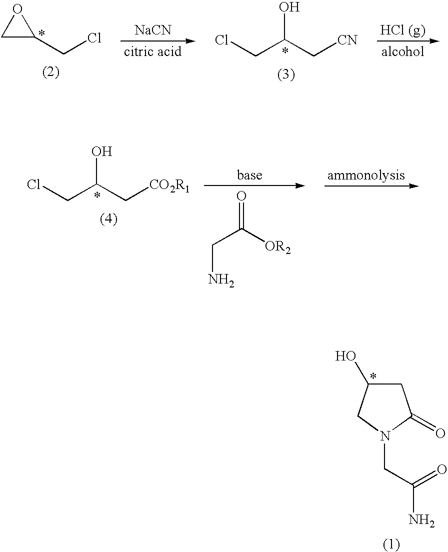
wherein R₁ and R₂ each independently represent alkyl groups having 1 to 4 carbon atoms and the asterisk represents a chiral center.

11. The process as set forth in claim 10, wherein R₁ and R₂ are each independently selected from the group consisting of methyl, ethyl, propyl, isopropyl, butyl, isobutyl and t-butyl.

12. The process as set forth in claim 11, wherein R₁ is ethyl and R₂ is methyl or ethyl.
